(19) United States
(12) Patent Application Publication
(10) Pub. No.: US 2026/0263545 A1
(43) Pub. Date: Sep. 10, 2026

(54) COMPOSITION FOR IMPROVING SKIN INFLAMMATION COMPRISING STEM EXTRACT OF GINSENG SPROUTS CULTIVATED BY LIGHT CONTROL

(71) Applicant: KOREA INSTITUTE OF SCIENCE AND TECHNOLOGY, Seoul (KR)

(72) Inventors: Jin-Chul KIM, Seoul (KR); Taek Sung LEE, Gangneung-si (KR); Hyoung Seok KIM, Gangneung-si (KR); Sang Hoon JUNG, Gangneung-si (KR); Jin-woo KIM, Gangneung-si (KR); YunHee SHIN, Gangneung-si (KR)

(73) Assignee: KOREA INSTITUTE OF SCIENCE AND TECHNOLOGY, Seoul (KR)

(21) Appl. No.: 19/365,994

(22) Filed: Oct. 22, 2025

(30) Foreign Application Priority Data
Mar. 5, 2025 (KR) 10-2025-0028074

(51) Int. Cl. A61K 36/258; A01G 9/24; A01G 22/25; A23L 33/105; A61P 17/00; A61P 29/00

(52) U.S. Cl. CPC A61K 36/258; A01G 9/249; A01G 22/25; A23L 33/105; A61P 17/00; A61P 29/00; A61K 2236/31; A61K 2236/33; A61K 2236/331; A61K 2236/333

(57) ABSTRACT

The present invention relates to a method of cultivating ginseng sprouts according to photoperiod control, and to a composition for preventing, ameliorating or treating skin inflammation such as atopic dermatitis, which includes a stem extract of ginseng sprouts cultivated thereby. The method of cultivating ginseng sprouts according to the present invention is particularly characterized in that the antioxidant and anti-atopic activities included in the ginseng sprout stem are increased by photoperiod control.

### CROSS-REFERENCE TO RELATED APPLICATION

This application claims priority to and the benefit of Korean Patent Application No. 2025-0028074, filed on Mar. 5, 2025, the disclosure of which is incorporated herein by reference in its entirety.

### BACKGROUND

### 1. Field of the Invention

The present invention relates to a method of cultivating ginseng for increasing the content of an active ingredient having an anti-inflammatory effect in the stems of ginseng sprouts according to photoperiod control, and to a composition for preventing, ameliorating, or treating skin inflammation including a stem extract of the ginseng sprouts.

### 2. Discussion of Related Art

Panax ginseng is a representative medical herb, and is a shade-loving plant belonging to the Araliaceae family. It is known to have various medicinal effects, such as immune-boosting, anticancer, blood sugar lowering, anti-inflammatory, and antioxidant activities, and many studies have been conducted to maximize the medicinal ingredients through various processing methods. In particular, Panax ginseng is rich in saponin, and the saponin components of Panax ginseng are mainly concentrated in the ginseng root, so the root part has been mainly used as the medicinal herb.

Research on the efficacy of natural medicinal herbs such as Panax ginseng is being conducted in various fields. In particular, there are attempts to utilize the natural medicinal herbs for the prevention or treatment of systemic diseases such as atopy. Atopy is an intractable disease that causes severe itching and skin inflammation. In the past 10 years, the number of patients with atopic dermatitis has been estimated to be over 950,000. In particular, it is known that 20% of children have an atopic disease. The market for treatments associated with atopic dermatitis has been known to have an annual growth rate of 6.8% from 2019 to 2024, and the medical expenses for atopic dermatitis treatment have also been steadily increasing at an annual average of over 21% for 5 years.

In particular, steroids, calcineurin inhibitors, and antihistamines are mainly used as the treatments for atopic dermatitis. However, topical steroids may cause side effects such as skin atrophy and telangiectasia, calcineurin inhibitors may also cause a burning sensation on the skin as a side effect, and antihistamines have the disadvantage of not being very effective. New antibody drugs have also been used, but they may cause conjunctivitis, keratitis, and the like, and have the problem of being very expensive. Also, due to the recent phenomenon of avoiding chemical substances, there is a growing tendency to prefer treatments using natural products that have excellent therapeutic effects and fewer side effects, but these natural products are generally expensive and have the problem of poor supply of raw materials.

### SUMMARY OF THE INVENTION

Therefore, the present inventors have conducted research on natural substances that have few side effects and excellent therapeutic effects and are easy to obtain as a treatment for skin inflammation such as atopy, and found that the stem of ginseng sprouts contains phenols and flavonoids, which have anti-inflammatory effects, and that the content of active ingredients contained in the stem of ginseng sprouts is maximized under specific light conditions. Therefore, the present invention has been completed on the basis of these findings.

Accordingly, the present invention is directed to providing a method of cultivating ginseng sprouts so as to increase the content of phenol or flavonoid components in the stem of ginseng sprouts.

Also, the present invention is directed to providing a pharmaceutical composition for preventing or treating skin inflammation, which includes a stem extract of the ginseng sprouts cultivated through the cultivation method.

In addition, the present invention is directed to providing a quasi-drug composition for preventing or ameliorating skin inflammation, which includes a stem extract of the ginseng sprouts cultivated through the cultivation method.

Further, the present invention is directed to providing a health functional food composition for preventing or ameliorating skin inflammation, which includes a stem extract of the ginseng sprouts cultivated through the cultivation method.

According to an aspect of the present invention, there is provided a method of cultivating ginseng sprouts so as to increase the content of phenol or flavonoid components in the stem of ginseng sprouts.

According to another aspect of the present invention, there is provided a pharmaceutical composition for preventing or treating skin inflammation, which includes a stem extract of the ginseng sprouts cultivated through the cultivation method.

According to still another aspect of the present invention, there is provided a quasi-drug composition for preventing or ameliorating skin inflammation, which includes a stem extract of the ginseng sprouts cultivated through the cultivation method.

According to yet another aspect of the present invention, there is provided a health functional food composition for preventing or ameliorating skin inflammation, which includes a stem extract of the ginseng sprouts cultivated through the cultivation method.

Hereinafter, the present invention will be described in detail.

According to one aspect of the present invention, the present invention is directed to a method of cultivating ginseng sprouts so as to increase the content of phenol or flavonoid components in the stem of ginseng sprouts.

The ginseng sprouts refers to ginseng seedlings that are one to two years old and grown for less than 8 weeks, preferably less than 6 weeks.

A method of cultivating ginseng sprouts according to the present invention includes the following steps:
1) subjecting seedlings of Panax ginseng to dark treatment and transplanting the seedlings of Panax ginseng into soil; and
2) maintaining light/dark cycles by repeating a light cycle of irradiating light having a light intensity of 50 PPFD to 250 PPFD at a temperature of 20 to 25° C. for 12 hours and a dark cycle of blocking light at a temperature of 15 to 20° C. for 12 hours.

Also, the carbon dioxide concentration may be maintained at 400 to 450 μmol·mol⁻¹ during the cultivation, and the cultivation period is 8 weeks, preferably less than 6 weeks.

When the ginseng sprouts are cultivated, the cultivation method includes a process of maintaining and repeating a light cycle and a dark cycle for a certain period of time. In this case, the light cycle is characterized by irradiating light having a light intensity of 50 PPFD to 250 PPFD, preferably a light intensity of 70 PPFD to 220 PPFD, and more preferably a light intensity of 200 PPFD or more.

The ginseng sprouts cultivated through the cultivation method of the present invention has the characteristic of increased phenol and flavonoid contents in the stem. The ginseng sprout “stem” refers to the part of the ginseng sprout excluding the underground part, leaves, and flowers. The ginseng sprout of the present invention has the characteristic of phenol and flavonoid contents in the stem being increased by 10 times or more, preferably 5 times or more, compared to typical ginseng sprouts.

The present invention is also directed to a pharmaceutical composition for preventing or treating skin inflammation, which includes a stem extract of the ginseng sprouts cultivated by the method.

In the present invention, “skin inflammation” is an immune response of the skin caused by various causes including atopy, and is an inflammatory response induced in skin tissue due to external stimuli, allergens, infections, autoimmune reactions, and the like. Skin inflammation is mainly accompanied by symptoms such as redness, swelling, itching, rashes, pain, and the like. In severe cases, the skin inflammation may progress to a chronic condition with skin barrier damage. In particular, atopic dermatitis is a representative skin inflammatory disease, and may cause a decline in skin barrier function and an excessive immune response due to the combined effects of genetic and environmental factors.

In one embodiment, the ginseng sprout stem extract of the present invention has the effect of alleviating and improving all symptoms caused by atopic dermatitis, particularly through a strong antioxidant effect, and inhibiting the expression of genes associated with atopic dermatitis.

The ginseng sprout stem extract of the present invention may be extracted by a known extraction method, and the present invention is not limited thereto. The extraction method may be selected from any one or more extraction methods selected from the group consisting of reduced-pressure high-temperature extraction, boiling extraction, reflux extraction, hot water extraction, cold immersion extraction, room-temperature extraction, ultrasonic extraction, or steam extraction.

The extract may use various solvents as an extraction solvent. In particular, water, a C₁₋₄ lower alcohol, or a mixed solvent thereof may be used. Specifically, water, ethanol, methanol, or a mixed solvent of water and ethanol may be selected and used. Preferably, the extract may be an ethanol extract.

In the present invention, the term “prevention” refers to any action capable of suppressing or delaying the onset of atopic dermatitis by administering the pharmaceutical composition of the present invention.

Also, the term “treatment” in the present specification refers to any action that ameliorates or beneficially changes symptoms by administering the pharmaceutical composition according to the present invention.

The pharmaceutical composition of the present invention may be formulated in the form of oral dosage forms such as powders, granules, tablets, capsules, suspensions, emulsions, syrups, aerosols, and the like, preparations for external use, suppositories, and sterile injectable solutions, and used according to conventional methods, and may further include carriers or excipients necessary for the formulation. Pharmaceutically acceptable carriers, excipients, and diluents that may be further included along with the active ingredient include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, magnesium stearate, mineral oil, and the like. When formulating, the pharmaceutical composition is usually prepared using commonly used diluents or excipients such as fillers, bulking agents, binders, wetting agents, disintegrants, surfactants, and the like.

For example, solid preparations for oral administration include tablets, pills, powders, granules, capsules, and the like, and these solid preparations are prepared by mixing the extract or compound with at least one excipient such as cotton, starch, calcium carbonate, sucrose or lactose, gelatin, and the like. In addition to simple excipients, lubricants such as magnesium stearate and talc are also used. Liquid preparations for oral administration include suspensions, oral solutions, emulsions, syrups, and the like. In addition to commonly used simple diluents such as water and liquid paraffin, various excipients such as wetting agents, sweeteners, flavoring agents, preservatives, and the like may be included.

Preparations for parenteral administration include sterile aqueous solutions, non-aqueous solvents, suspensions, emulsions, lyophilized preparations, and suppositories. The non-aqueous solvents and suspensions may include propylene glycol, polyethylene glycol, vegetable oils such as olive oil, injectable esters such as ethyl oleate, and the like. Witepsol, Macrogol, Tween 61, cacao butter, laurin butter, glycerogelatin, and the like may be used as suppository bases.

The pharmaceutical composition of the present invention may be administered orally or parenterally (by intravenous injection, subcutaneous, intraperitoneal or topical application) depending on the desired method, and the dosage varies depending on the patient's condition and weight, the severity of a disease, the form of a drug, the route and time of administration, and the like, and may be selected in an appropriate form by a person skilled in the art.

The pharmaceutical composition of the present invention is administered in a pharmaceutically effective amount. In the present invention, the term “pharmaceutically effective amount” refers to a reasonable amount applicable to medical treatment, that is, an amount sufficient to treat a disease. In this case, the standard for the pharmaceutically effective amount may be determined depending on the type of a patient's disease, the severity of the disease, the activity of a drug, the sensitivity to the drug, the time of administration, the route of administration, and the excretion rate, the duration of treatment, components used concurrently, and other factors. The pharmaceutical composition of the present invention may be administered as a separate therapeutic agent or in combination with other therapeutic agents, and may be administered sequentially or simultaneously with conventional therapeutic agents. The dosage may be determined at a level that may minimize side effects in consideration of all of the above factors, and thus may be easily determined by a person skilled in the art. Specifically, the dosage of the pharmaceutical composition may vary depending on the patient's age, weight, severity, sex, and the like. In this case, the pharmaceutical composition may generally be administered in an amount of 0.001 to 150 mg per 1 kg of body weight, more preferably 0.01 to 100 mg, once to three times a day, every day or every other day. However, this is exemplary, and the dosage may be set differently when necessary.

The present invention is also directed to a quasi-drug composition for preventing or ameliorating skin inflammation, which includes a stem extract of the ginseng sprouts cultivated by the method.

The quasi-drug composition of the present invention includes preparations for external use, powders, sterilizing disinfectants, detergents, toothpaste, ointments, lotions, preparations for internal use (vitamin/mineral preparations, tonics), wet tissues, spray patches, bandages or patches, and the like, but the present invention is not particularly limited thereto. The formulation method, dosage, method of use, components, and the like of a quasi-drug may be appropriately selected from common techniques known in the technical field of the present invention.

Also, the quasi-drug composition of the present invention may be a composition for external skin application for preventing or ameliorating atopic dermatitis. Here, in addition to the ginseng sprout stem extract, the composition for external skin application may further include an adjuvant commonly used in the field of dermatology, such as a fatty substance, an organic solvent, a solubilizing agent, a thickening and gelling agent, a softening agent, an antioxidant, a suspending agent, a stabilizing agent, a foaming agent, a fragrance, a surfactant, water, an ionic or non-ionic emulsifying agent, a filler, a metal ion sequestering/chelating agent, a preservative, a vitamin, a blocking agent, a humectant, an essential oil, a dye, a pigment, a hydrophilic or lipophilic active agent, a lipid vesicle, or any other ingredients commonly used in external skin applications. Also, the components may be introduced in an amount commonly used in the field of dermatology.

Further, the present invention is directed to a health functional food composition for preventing or ameliorating skin inflammation, which includes a stem extract of the ginseng sprouts cultivated by the method.

The term “health functional food” refers to a food that is manufactured and processed using raw materials or components having functionality useful to the human body according to Act No. 6727 on Health Functional Foods, and the term “functional” means that it is consumed for the purpose of regulating nutrients for the structure and function of the human body or obtaining useful effects for health purposes, such as physiological effects and the like.

The food or health functional food of the present invention may be manufactured and processed into pharmaceutical dosage forms such as powders, granules, tablets, capsules, pills, suspensions, emulsions, syrups, and the like, or health functional foods such as tea bags, infusions, beverages, candies, jellies, gums, and the like for the purpose of preventing or ameliorating skin inflammation.

The food or health functional food composition of the present invention may be used as a food additive, and may be manufactured into products alone or in combination with other components. Also, nutrients, vitamins, electrolytes, flavoring agents, coloring and enhancing agents, pectic acid and salts thereof, alginic acid and salts thereof, organic acids, protective colloid thickening agents, pH regulators, stabilizing agents, preservatives, glycerin, alcohol, carbonating agents used in carbonated beverages, and the like may be included. The components may be used alone or in combination, and may be combined and used in an appropriate amount.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic diagram showing the cultivation conditions (light intensity of 70 PPFD, 200 PPFD) of ginseng sprouts with an increased content of active ingredients in the stem and the cultivation process to which these conditions are applied;

FIG. 2 shows the results of confirming the antioxidant effect of the stems of ginseng sprouts cultivated under each light condition;

FIG. 3 shows the results of comparing the total phenol content (A) and total flavonoid content (B) of the stems of ginseng sprouts cultivated under each light condition; and

FIG. 4 shows the results of confirming the effect of suppressing the expression of atopic-related genes using a stem extract of the ginseng sprouts cultivated under each light condition.

### DETAILED DESCRIPTION OF EXEMPLARY EMBODIMENTS

Hereinafter, examples will be described in detail to specifically explain the present specification. However, it should be understood that the examples according to the present specification may be modified into various different forms, and the scope of the present specification is not limited to the examples described below. The examples of the present specification are provided to more completely explain the present specification to a person having ordinary skill in the art.

### EXAMPLE 1

### Cultivation of Ginseng Sprouts and Preparation of Extract

### 1-1. Cultivation of Ginseng Sprouts According to Light Intensity

One-year-old ginseng seedlings purchased from Jangseong-gun, Jeollanam-do were subjected to dark treatment to induce new shoots, and then transplanted into oval pots using ginseng-specific bed soil (ON Ginseng Bed Soil, Oncelling, Korea). This experiment was performed at the XYZ Cultivation Group located in the research building of the Korea Institute of Science and Technology (KIST), and ginseng sprouts was cultivated under the cultivation conditions of an indoor temperature of 23° C./18° C. (light/dark), a humidity of 46%, a carbon dioxide concentration of 440 μmol·mol⁻¹, a 12 h/12 h (light/dark) cycle, and 500 mL irrigation (once/7 days) for 6 weeks. At this time, the cultivation was performed under white light, and the light intensity used was 70 μmol·m⁻²·S⁻¹ (70 PPFD, 1000 light condition) and 200 μmol·m⁻²·S⁻¹ (200 PPFD, 1002 light condition).

### 1-2. Preparation of Ethanol Extract of Stems of Ginseng Sprouts

The stem extract of the ginseng sprouts was prepared according to the following procedure. The stems of ginseng sprouts were dried at 50° C. for 5 days. Each dried sample was mixed with 50% ethyl alcohol (dry sample: 50% ethyl alcohol=1:10 (w/v)). Thereafter, reflux extraction was performed at 25° C. for 24 hours, and this process was repeated twice. Each extract was filtered through 0.45 μm filter paper, concentrated under reduced pressure using a 40° C. constant-temperature water bath (Buchi, Flawil, Switzerland) and a rotary evaporator (Buchi), and then freeze-dried. The prepared extracts were stored in a freezer (−20° C.).

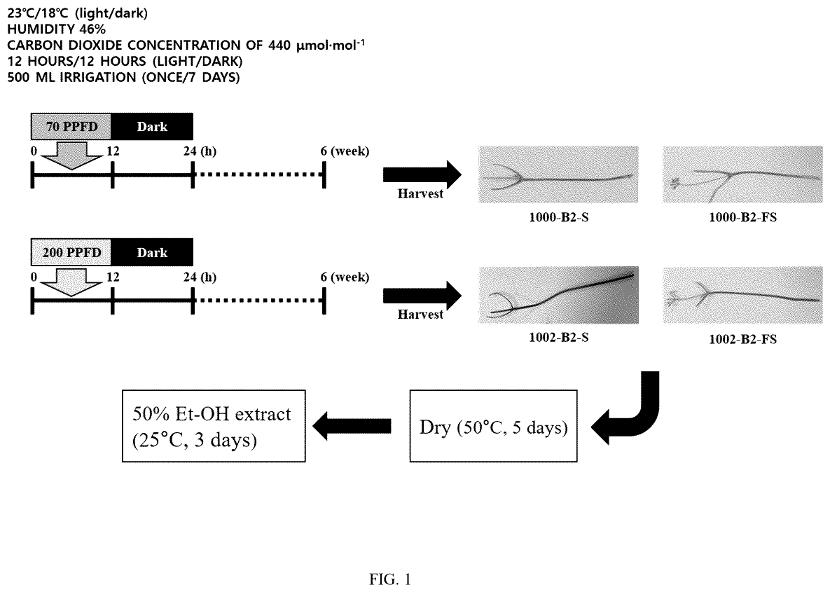

The process of cultivating ginseng sprouts and the process of preparing the extract are shown in FIG. 1.

### EXPERIMENTAL EXAMPLE 1

### Confirmation of Antioxidant Activity

In Example 1 above, in order to confirm the antioxidant activity of each type of ginseng sprout stem (1000-B2-S, 1002-B2-S) cultivated under low-light and high-light intensity conditions, an experiment was conducted to measure DPPH radical scavenging activity and ABTS radical scavenging activity. To confirm whether the antioxidant effect was the effect of the active ingredients included only in the stem, the same experiment was conducted on the stems (1000-B2-FS, 1002-B2-FS) without removing the flowers.

First, the DPPH radical scavenging activity was measured by adding each ginseng sprout stem extract of Example 1-2 to a 2,2-diphenyl-1-picrylhydrazyl (DPPH) solution, allowing it to react for a certain period of time, and then measuring the absorbance at 517 nm to evaluate the radical scavenging activity.

Also, a 2,2′-azinobis-(3-ethylbenzothiazoline-6-sulfonic acid) (ABTS) solution was reacted with potassium persulfate to generate ABTS+radicals, and then the sample was added and allowed to react. Then, the absorbance was measured at 732 nm to evaluate the radical scavenging activity.

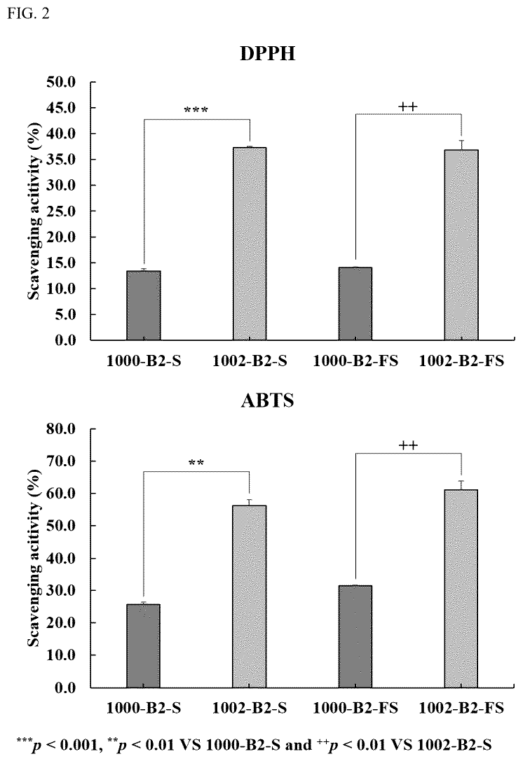

As the results of the above evaluation, as shown in FIG. 2, it was confirmed that the DPPH radical and ABTS radical scavenging activities in the extracts of stems of ginseng sprouts cultivated by treatment with high-intensity light were significantly improved, and no significant difference was observed between the stems including and not including flowers.

### EXPERIMENTAL EXAMPLE 2

### Comparison of Phenol and Flavonoid Contents

In Example 1 above, the total phenol content and flavonoid content were confirmed for each stem extract of the ginseng sprouts (1000-B2-S, 1002-B2-S, 1000-B2-FS, and 1002-B2-FS) cultivated under the low-light and high-light intensity conditions.

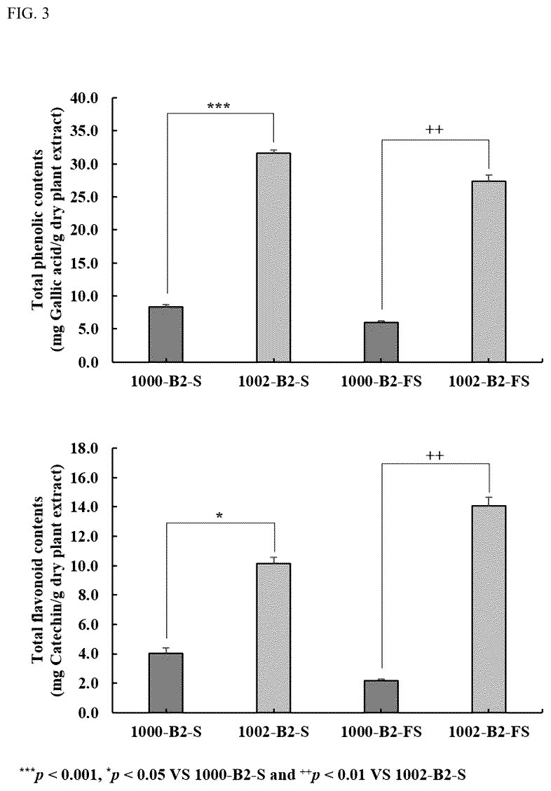

As shown in FIG. 3, it can be seen that the total phenol content and flavonoid content increased in the stem extracts of ginseng sprouts cultivated by treatment with high-intensity light.

### EXPERIMENTAL EXAMPLE 3

### Comparison of Expression of Genes Associated with Atopic Dermatitis

In Example 1 above, the effects of each stem extract of the ginseng sprouts (1000-B2-S, 1002-B2-S, 1000-B2-FS, and 1002-B2-FS) cultivated under low-light and high-light intensity conditions on atopic dermatitis were confirmed as follows.

HaCaT cells were cultured in a DMEM medium supplemented with 10% FBS and 1% penicillin/streptomycin under 37° C. and 5% CO₂ conditions, and the cultured cells were seeded into 6-well or 12-well plates, and then treated with 10 ng/mL of each of TNF-α and IFN-γ to induce inflammation. The cell culture medium was treated with the stem extracts of the ginseng sprouts (1000-B2-S, 1002-B2-S, 1000-B2-FS, and 1002-B2-FS), and the RNA expression levels of inflammatory cytokines such as CCL-4, IL-4, IL-6, IL-8, and IL-33, which are markers associated with atopy, were compared.

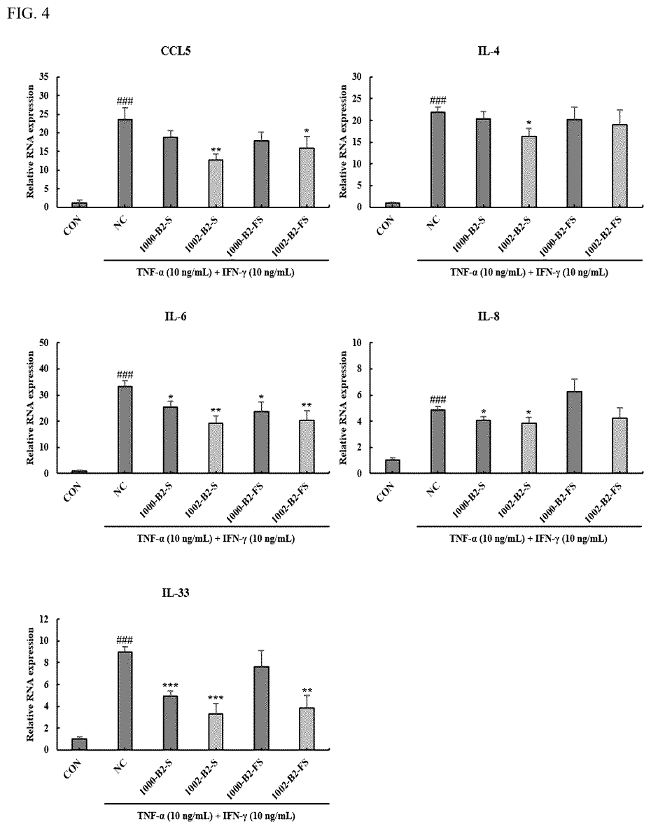

As a result, as shown in FIG. 4, it can be seen that when the cells were treated with the stem extracts of the ginseng sprouts (1000-B2-S, 1002-B2-S, 1000-B2-FS, and 1002-B2-FS) after inducing inflammation, the relative RNA expression levels of inflammatory cytokines were reduced compared to the untreated group (NC).

According to the present invention, it was confirmed that the content of active ingredients such as phenols or flavonoids having anti-inflammatory effects can be increased in the stems of ginseng sprouts by limiting the photoperiod and cultivation conditions during the cultivation of ginseng sprouts. Therefore, since the ginseng sprout stems have strong antioxidant activity and the effect of suppressing and regulating the expression of various genetic factors associated with atopic dermatitis, the ginseng sprout stems can be used for the purpose of treating or preventing skin inflammation such as atopic dermatitis.

The present invention has been described and shown with reference to the preferred embodiments thereof. Those skilled in the art to which the present invention pertains will appreciate that the present invention may be implemented in modified forms without departing from the essential characteristics of the present invention. Therefore, the disclosed embodiments are to be considered in all respects as illustrative and not restrictive. The scope of the invention is shown in the claims rather than in the foregoing description, and all differences within the scope equivalent to the above-described embodiments should be construed as being included in the present invention.

What is claimed is:

1. A method of cultivating ginseng sprouts, comprising:
1) subjecting ginseng sprout seedlings to dark treatment and transplanting the ginseng sprout seedlings into soil; and
2) maintaining light/dark cycles by repeating a light cycle of irradiating light having a light intensity of 50 PPFD to 250 PPFD at a temperature of 20 to 25° C. for 12 hours and a dark cycle of blocking light at a temperature of 15 to 20° C. for 12 hours.

2. The method of claim 1, wherein the carbon dioxide concentration is maintained at 400 to 450 μmol·mol⁻¹ during the cultivation of ginseng sprouts.

3. The method of claim 1, wherein the ginseng sprout cultivation period is within 8 weeks.

4. A pharmaceutical composition for preventing or ameliorating skin inflammation, comprising a stem extract of the ginseng sprouts cultivated by the method of cultivating ginseng sprouts of claim 1.

5. The pharmaceutical composition of claim 4, wherein the ginseng sprout stem extract is extracted using water, a C₁₋₄ lower alcohol, or a mixed solvent thereof.

6. The pharmaceutical composition of claim 4, wherein the skin inflammation includes atopic dermatitis.

7. A quasi-drug composition for preventing or ameliorating skin inflammation, comprising a stem extract of the ginseng sprouts cultivated by the method of cultivating ginseng sprouts of claim 1.

8. The quasi-drug composition of claim 7, wherein the ginseng sprout stem extract is extracted using water, a C₁₋₄ lower alcohol, or a mixed solvent thereof.

9. The quasi-drug composition of claim 7, wherein the skin inflammation includes atopic dermatitis.

10. A health functional food composition for preventing or ameliorating skin inflammation, comprising a stem extract of the ginseng sprouts cultivated by the method of cultivating ginseng sprouts of claim 1.

11. The health functional food composition of claim 10, wherein the ginseng sprout stem extract is extracted using water, a C₁₋₄ lower alcohol, or a mixed solvent thereof.

12. The health functional food composition of claim 10, wherein the skin inflammation includes atopic dermatitis.

13. A method for preventing or treating skin inflammation, comprising administering to a subject in need thereof a stem extract of ginseng sprouts cultivated by the method of claim 1.

14. The method of claim 13, wherein the stem extract is administered topically or orally.

15. The method of claim 13, wherein the stem extract is extracted using water, a C1-C4 lower alcohol, or a mixture thereof.

16. The method of claim 13, wherein the skin inflammation comprises atopic dermatitis.
